(19) United States
(12) Patent Application Publication
(10) Pub. No.: US 2015/0258273 A1
(43) Pub. Date: Sep. 17, 2015

(54) Electrochemically-Actuated Microfluidic Devices

(71) Applicants: Forrest W. Payne, Fayetteville, AR (US); Sai Ramamurthy Kumar, Johns Creek, GA (US); Christine E. Evans, North Liberty, IA (US); Anna Washburn, Elkins, AR (US); Andy M. Dunn, Santa Monica, CA (US); Brian Young, Bloomington, IN (US); Joe Bruton, Fayetteville, AR (US); Champak Das, Fayetteville, AR (US); Kavita M. Jeerage, Denver, CO (US); Carl A. Koval, Arvada, CO (US); Richard D. Noble, Boulder, CO (US)

(72) Inventors: Forrest W. Payne, Fayetteville, AR (US); Sai Ramamurthy Kumar, Johns Creek, GA (US); Christine E. Evans, North Liberty, IA (US); Anna Washburn, Elkins, AR (US); Andy M. Dunn, Santa Monica, CA (US); Brian Young, Bloomington, IN (US); Joe Bruton, Fayetteville, AR (US); Champak Das, Fayetteville, AR (US); Kavita M. Jeerage, Denver, CO (US); Carl A. Koval, Arvada, CO (US); Richard D. Noble, Boulder, CO (US)

(21) Appl. No.: 13/598,351

(22) Filed: Aug. 29, 2012

Related U.S. Application Data
(60) Provisional application No. 61/529,578, filed on Aug. 31, 2011.

(51) Int. Cl. A61M 5/142; F15C 3/04; A61M 5/145

(52) U.S. Cl. CPC A61M 5/14224; A61M 5/14593; A61M 5/14244; F15C 3/04; A61M 2005/14204; A61M 2005/14513; A61M 2205/0244

(57) ABSTRACT

Electrochemical actuation is disclosed for fluid movement and flow control in microfluidic devices, allowing for miniaturization, minimal power requirements, single-use disposability and engineering of small, complex fluidic networks. In one embodiment, a single-dose fluid delivery device is operable to deliver a bolus dose, in a single extended stroke or in multiple repeated doses. The device uses three electrochemically-actuated chambers, two of the chambers operating as inlet/outlet valves for the device and a third providing both a temporary containment and pumping action. By sequential manipulation of the fluid pressure in the three chambers, fluids may be delivered in precise quantities by the device.

### CROSS-REFERENCE TO RELATED APPLICATIONS

This application claims priority from U.S. provisional patent application Ser. No. 61/529,578, filed on Aug. 31, 2011, and entitled “Self-Valving Electrochemical Pump.” Such application is incorporated herein by reference.

### STATEMENT REGARDING FEDERALLY SPONSORED RESEARCH OR DEVELOPMENT

This invention was made with Government support under the terms of Grant No. 0848253, awarded by the National Science Foundation and under the terms of Contract No. W81XWH-09-1-0523, awarded by the Department of Defense. The Government has certain rights to the invention.

### BACKGROUND OF THE INVENTION

The invention is in the field of electrochemical actuation of microfluidic devices, including pumping action, valving action, flow rate control, flow direction control, dispense control, sample introduction, and amplification of volumetric flow rate or pressure and solution storage.

Microfluidic devices have great promise to revolutionize a wide range of detection and fluid delivery applications, including environmental testing, product quality control and clinical diagnostics/drug delivery. Microfluidics can scale down the size, power requirements, reagent use and waste production of assays and can control the delivery of fluids in minute quantities. Furthermore, with the applications described as examples in this patent realized, new uses for microfluidic systems are likely to ensue.

There are many research and product development teams around the world that are studying miniaturized assay platforms and detection technologies. However, the full potential of these technologies will not be realized until entire microfluidic systems are envisioned and engineered. All aspects of a microfluidic device (such as pumps, valves, chambers, amplifiers, fluidic paths and more) must meet the mandates of small footprint, low power requirements and precise/reproducible control.

The inventors hereof have recognized that electrochemical actuators may be key components of microfluidic devices. The use of controlled electrical flow to drive a chemical reaction that then actuates a device component would allow for miniaturization of all device aspects. The coordinated use of these components would allow for actuation and precise control of complicated flow regimes in microfluidic devices. Microfluidic devices could make use of the electrochemical actuators described herein, along with magnetic, magnetohydrodynamic, ultrasonic, electrohydrodynamic, electroosmotic, piezoelectric and electrokinetic actuation mechanisms.

The inventors hereof have recognized that insulin delivery would be an application of great value for the fluid delivery devices described herein according to preferred embodiments of the present invention. The delivery of insulin to diabetic patients in precisely programmed basal/bolus doses may be better than multiple daily injections. The benefits of such controlled delivery include tighter glycemic control with less fluctuation, greater control of nighttime hypoglycemia and post-meal hyperglycemia, lower total insulin requirements, and the ability to precisely track insulin dosage. Patients prefer wearable drug delivery devices due to the more convenient and unobtrusive lifestyle they offer, flexibility in eating schedules, assistance in dose calculation/delivery, and data storage. The inventors have further recognized that when integrated with a glucose sensor, the result of such a system could be an automatic feedback basal/bolus control for proactive prevention of diabetic complications. The resulting “artificial pancreas” system according to a preferred embodiment would be a major revolution in the treatment of diabetes, since it would closely mimic the function of the natural pancreas. Closed loop drug delivery and sensor monitoring, safety from mechanical and non-mechanical failures, and programmed, heuristic insulin delivery are some of the advantages that the inventors foresee using such a device.

Currently, there are many commercial insulin delivery systems available in the marketplace. Each of those systems, however, suffers from certain disadvantages that have prevented widespread adoption of these systems in place of multiple daily insulin injections. Currently available wearable insulin delivery devices are expensive, require frequent replacement, suffer from mechanical failures, or require management of multiple components and complex operations. What is desired then is a delivery device that overcomes the shortcomings of existing commercial devices, specifically seeking to realize low cost, optimal performance, small and ergonomic design, minimal power usage, and multiple level fail-safety, all of which are enabled by the appropriate combination of electrochemically actuated components, as described here.

References and information mentioned in this background section are not admitted to be prior art with respect to the present invention.

### SUMMARY OF THE INVENTION

The present invention is directed to the coordinated use of electrochemical pumps and valves, which allows for the creation of small, elegant, complex, inexpensive, microfluidic devices with very low power requirements. These devices have the greatest potential to revolutionize medical diagnostics and treatments, in particular for applications in low-resources settings or those that would benefit from rapid diagnosis/application. Furthermore, the control systems for these devices are minimal and require very low voltages, allowing for on-board and/or remote controlled operation. A number of embodiments are detailed herein that show the broad applicability of this invention. These embodiments are not meant in any way to limit the scope of this invention.

In certain aspects, the present invention is directed to a fluid delivery device including at least one electrochemical pump/pumping method and at least one electrochemical valve/valving method that controls movement of a fluid from one location to another. Such fluids may include, without limitation, drug delivery fluids. For example, the invention could be used to move a liquid from an external reservoir into an application such as delivering a drug to a patient. Two of the many applications for the present invention include the delivery of insulin to diabetic patients and delivery of medicines to the eyes of humans or animals. The elimination of the need for mechanical pumps and valves reduces the overall size, complexity, and power consumption of the device, which allows for miniaturization of the whole device to the desired extent. In certain embodiments, the device may also act as a closed valve when not in use, preventing fluid from inadvertently passing from the reservoir to the application. In this manner, certain embodiments of the present invention provide a failsafe mechanism for preventing accidental drug delivery. The invention in certain embodiments may allow for very fine-tuned control of fluid delivery in either continuous or bolus doses. Using insulin as an example, the device could be used to very closely mimic natural insulin delivery rates from a healthy pancreas.

In a first aspect, the invention is directed to a device for the directional delivery of a fluid, comprising a pump module comprising an electrochemical actuator configured to selectively apply a pressure within the pump module, an inlet valve comprising an electrochemical actuator configured to selectively apply a pressure within the inlet valve, an outlet valve comprising an electrochemical actuator configured to selectively apply a pressure within the outlet valve, an external reservoir in fluid communication with the inlet valve, an application in fluid communication with the outlet valve, a first channel fluidically connecting the inlet valve and the pump module, a second channel fluidically connecting the outlet valve and the pump module, an inlet diaphragm positioned within the inlet valve, wherein the inlet diaphragm is flexible to assume one of a flexed state and an unflexed rest state, and wherein the inlet diaphragm is positioned to block flow of the fluid between the external reservoir and the first channel when the diaphragm is in the unflexed rest state, and to allow flow of the fluid between the external reservoir and the first channel when the inlet diaphragm is in the flexed state, an outlet diaphragm positioned within the outlet valve, wherein the outlet diaphragm is flexible to assume one of a flexed state and an unflexed rest state, and wherein the outlet diaphragm is positioned to block flow of the fluid between the second channel and the application when the diaphragm is in the unflexed rest state, and to allow flow of the fluid between the second channel and the application when the diaphragm is in the flexed state, and an internal reservoir diaphragm positioned within the pump module, wherein the internal reservoir diaphragm is flexible to assume one of a flexed state and an unflexed rest state, and wherein the internal reservoir diaphragm is positioned to receive flow of the liquid from the first channel into a reservoir formed by the internal reservoir diaphragm when the internal reservoir diaphragm is in the flexed state, and to discharge the fluid from the reservoir into the second channel when the internal reservoir diaphragm moves from the flexed state to the unflexed state.

In a second aspect, the invention is directed to a method of directionally pumping a fluid using a pump, wherein the pump comprises a pump module, an inlet valve, an outlet valve, an external reservoir inlet in fluid communication with the inlet valve, an application outlet in fluid communication with the outlet valve, an inlet channel fluidically connecting the inlet valve and the pump module, an outlet channel fluidically connecting the outlet valve and the pump module, an inlet diaphragm positioned within the inlet valve, an outlet diaphragm positioned within the outlet valve, and an internal reservoir diaphragm positioned within the pump module, the method comprising the steps of activating the inlet valve to create a force in a first direction, whereby the inlet diaphragm flexes in the first direction to form an inlet passage between the external reservoir inlet and the inlet channel, activating the pump module to create a force in the first direction, whereby the internal reservoir diaphragm flexes in the first direction to form an internal reservoir, and thereby drawing the fluid from the external reservoir inlet into the internal reservoir, activating the inlet valve to create a force in a second direction, whereby the inlet diaphragm returns to a rest state to close the inlet passage between the external reservoir inlet and the inlet channel, activating the outlet valve to create a force in the second direction, whereby the outlet diaphragm flexes in the second direction to form an outlet passage between the outlet channel and the application outlet, and activating the pump module to create a force in the second direction, whereby the internal reservoir diaphragm flexes in the second direction, thereby forcing fluid from the internal reservoir through the outlet channel and into the application outlet.

In a third aspect, the invention is directed to a method of directionally pumping a fluid using a pump, wherein the pump comprises a pump module, an inlet valve, an outlet valve, a first external reservoir inlet and a second external reservoir inlet in fluid communication with the inlet valve, a first application outlet and a second application outlet in fluid communication with the outlet valve, a first inlet channel and a second inlet channel fluidically connecting the inlet valve and the pump module, a first outlet channel and a second outlet channel fluidically connecting the outlet valve and the pump module, a first inlet diaphragm and a second inlet diaphragm positioned within the inlet valve, a first outlet diaphragm and a second outlet diaphragm positioned within the outlet valve, and a first internal reservoir diaphragm and a second internal reservoir diaphragm positioned within the pump module, the method comprising the steps of activating the inlet valve to create a force in a first direction, whereby the first inlet diaphragm flexes in the first direction to form a first inlet passage between the first external reservoir inlet and the first inlet channel, and the second inlet diaphragm flexes in the first direction to close a second inlet passage between the second external reservoir inlet and the second inlet channel, activating the pump module to create a force in the first direction, whereby the first internal reservoir diaphragm flexes in the first direction to form a first internal reservoir, and thereby drawing the fluid from the first external reservoir inlet into the first internal reservoir, activating the inlet valve to create a force in a second direction, whereby the first inlet diaphragm returns to a rest state to close the inlet passage between the first external reservoir inlet and the first inlet channel, and whereby the second inlet diaphragm flexes in the second direction to form a second inlet passage between the second external reservoir inlet and the second inlet channel, activating the outlet valve to create a force in the first direction, whereby the first outlet diaphragm flexes in the first direction to form a first outlet passage between the first outlet channel and the first application outlet, activating the pump module to create a force in a second direction, whereby the first internal reservoir diaphragm flexes in the second direction, thereby forcing fluid from the first internal reservoir through the first outlet channel and into the first application outlet, and whereby the second internal reservoir diaphragm flexes in the second direction, thereby drawing fluid from the second inlet channel into the second internal reservoir, activating the inlet valve to create a force in the first direction, whereby the first inlet diaphragm flexes in the first direction to open the first inlet passage between the first external reservoir inlet and the first inlet channel, and the second inlet diaphragm flexes in the first direction to close the second inlet passage between the second external reservoir inlet and the second inlet channel, activating the outlet valve to create a force in the second direction, whereby the first inlet valve diaphragm flexes in the second direction to close the first outlet passage between the first outlet channel and the first application outlet, and the second outlet diaphragm flexes in the second direction to open the second outlet passage between the second outlet channel and the second application outlet, and activating the pump module to create a force in the first direction, whereby the first internal reservoir diaphragm flexes in the first direction to open the first internal reservoir, and thereby drawing the fluid from the first external reservoir inlet into the first internal reservoir, and whereby the second internal reservoir diaphragm flexes in the first direction, thereby forcing fluid from the second internal reservoir through the second outlet channel and into the second application outlet.

In a fourth aspect, the invention is directed to an electrochemical actuator, comprising a semi-permeable membrane comprising a first and second side, a first pump body positioned adjacent to the first membrane side, a second pump body positioned adjacent to the second membrane side, a first diaphragm positioned adjacent to the first pump body opposite from the semi-permeable membrane, a second diaphragm positioned adjacent to the second pump body opposite from the semi-permeable membrane, a first pump cap positioned adjacent to the first diaphragm, and a second pump cap positioned adjacent to the second diaphragm.

In a fifth aspect, the invention is directed to a valve actuator in communication with a fluidic path, comprising an electrochemical pump comprising an elastomer, a mechanical valve in contact with the elastomer, whereby the mechanical valve is operable to open and close the fluidic path, and a force generator configured to generate a force capable of moving the mechanical valve against the elastomer in a direction opposite of a direction in which the elastomer expands as a result of operation of the electrochemical pump.

In a sixth aspect, the invention is directed to a fluid control mechanism, comprising a flexible fluid container comprising a volume, and an electrochemical actuator comprising an elastomer diaphragm, wherein the electrochemical actuator is configured to flex the elastomer diaphragm outward, and further wherein the elastomer diaphragm is in contact with the fluid container whereby outward flexing of the elastomer diaphragm reduces the volume of the fluid container.

In a seventh aspect, the invention is directed to an actuation device, comprising an electrochemical actuator comprising an elastomer diaphragm, wherein the electrochemical actuator is configured to flex the elastomer diaphragm outward and wherein the electrochemical actuator comprises a diaphragm cross-sectional area, a piston comprising a first and second section wherein the piston first section is connected to the elastomer diaphragm, and a flow channel sized to receive the second section of the piston.

In an eighth aspect, the invention is directed to a galvanic electrochemical actuator, comprising a first electrochemical cell half comprising an electrolyte and a cathode/anode, a second electrochemical cell half comprising the electrolyte and an anode/cathode, an ion-permeable membrane separating the first and second electrochemical cell halves, and an electrical connection between the cathode and anode, whereby an ion flux is generated through the ion permeable membrane.

The inventors have recognized numerous additional applications, including a single-dose fluid delivery device, a continuous-dose fluid delivery device, disposable, low-cost actuators, self-powered actuators, magnetic valve actuators, elastic valve actuators, programmable microfluidic chips, flow rate and fluid force amplification actuators and self-contained ELISA chips.

The inventors have further recognized that the small size, low power requirements and non-mechanical nature of electrochemical actuators according to various embodiments means that devices composed of these actuators could be small and complex and provide multiple layers of fail-safety. This safety feature would be particularly important if the fluid being delivered is a drug to a patient and would greatly reduce the possibility of overdose. Additionally, the inventors have recognized that devices based on electrochemical pumps and valves can operate at the pressures and flow rates required to run a sandwich ELISA. In another example, the inventors have recognized that the minimal power requirements needed to run an electrochemically-actuated device could be provided by conventional, battery or solar (for repeated operation) or by an additional chemical reaction (for one-time operation).

These and other objects, features, and advantages of the present invention will become better understood from a consideration of the following detailed description of the preferred embodiments and appended claims in conjunction with the drawings as described following.

### BRIEF DESCRIPTION OF THE DRAWINGS

FIG. 1A is a schematic diagram showing a first arrangement for an electrochemically-actuated fluid delivery device according to a preferred embodiment of the present invention.

FIG. 1B is a schematic diagram showing a second arrangement for an electrochemically-actuated fluid delivery device according to a preferred embodiment of the present invention.

FIG. 2A is a schematic diagram showing a single-dose directional fluid delivery device when at rest according to a preferred embodiment of the present invention.

FIG. 2B is a schematic diagram showing the single-dose directional fluid delivery device of FIG. 2A in a first step of an action sequence for directional fluid delivery according to a preferred embodiment of the present invention.

FIG. 2C is a schematic diagram showing the single-dose directional fluid delivery device of FIG. 2A in a second step of an action sequence for directional fluid delivery according to a preferred embodiment of the present invention.

FIG. 2D is a schematic diagram showing the single-dose directional fluid delivery device of FIG. 2A in a third step of an action sequence for directional fluid delivery according to a preferred embodiment of the present invention.

FIG. 2E is a schematic diagram showing the single-dose directional fluid delivery device of FIG. 2A in a fourth step of an action sequence for directional fluid delivery according to a preferred embodiment of the present invention.

FIG. 2F is a schematic diagram showing the single-dose directional fluid delivery device of FIG. 2A in a fifth step of an action sequence for directional fluid delivery according to a preferred embodiment of the present invention.

FIG. 2G is a schematic diagram showing the single-dose directional fluid delivery device of FIG. 2A in a sixth step of an action sequence for directional fluid delivery according to a preferred embodiment of the present invention.

FIG. 3A is a schematic diagram showing a continuous-dose directional fluid delivery device when at rest according to a preferred embodiment of the present invention.

FIG. 3B is a schematic diagram showing the continuous-dose directional fluid delivery device of FIG. 3A in a first step of an action sequence for directional fluid delivery according to a preferred embodiment of the present invention.

FIG. 3C is a schematic diagram showing the continuous-dose directional fluid delivery device of FIG. 3A in a second step of an action sequence for directional fluid delivery according to a preferred embodiment of the present invention.

FIG. 3D is a schematic diagram showing the continuous-dose directional fluid delivery device of FIG. 3A in a third step of an action sequence for directional fluid delivery according to a preferred embodiment of the present invention.

FIG. 4 is a graph showing quasi-continuous fluid delivery using an electrochemical actuator according to a preferred embodiment of the present invention.

FIG. 5 is a perspective exploded view of a molded electrochemical actuator body according to a preferred embodiment of the present invention.

FIG. 6 is a graph showing the electrochemical current produced by a potential applied using titanium electrodes according to a preferred embodiment of the present invention.

FIGS. 7A and 7B are schematics illustrating a preferred embodiment of a self-powered electrochemical actuator.

FIG. 8A is a graph showing flow rate produced using a self-powered electrochemical actuator according to a preferred embodiment of the present invention.

FIG. 8B is a graph showing dispense volume produced using a self-powered electrochemical actuator according to a preferred embodiment of the present invention.

FIG. 9 is a schematic depicting a magnetic valve actuator according to a preferred embodiment of the present invention.

FIG. 10 is a graph showing flow control enabled by a magnetic valve actuator.

FIG. 11 is a schematic depicting an electrochemical valve with elastic actuator being used for dispense/of fluid from a removable reservoir according to a preferred embodiment of the present invention.

FIG. 12 is a graph showing flow rates of fluid from a removable elastic valve actuator.

FIG. 13A is a schematic of in-channel pinch valve/pump actuators according to a preferred embodiment of the present invention.

FIG. 13B is one schematic of in-channel pinch valve actuators used to combine fluids in precisely controlled amounts according to a preferred embodiment of the present invention.

FIG. 13C is a schematic of in-channel pinch valve actuators used to make droplet mixtures in non-miscible solutions according to a preferred embodiment of the present invention.

FIG. 13D is a schematic of in-channel pinch valve actuators used to change direction of flow to induce mixing according to a preferred embodiment of the present invention.

FIG. 13E is a schematic of in-channel pinch valve actuators used for mixing in an ‘S’ bend according to a preferred embodiment of the present invention.

FIG. 14 is a schematic depicting a volume amplification actuator according to a preferred embodiment of the present invention.

FIG. 15 is a graph showing an increase in flow rate generated by a volume amplification actuator.

FIG. 16 is a schematic depicting a pressure amplification actuator according to a preferred embodiment of the present invention.

FIG. 17 is a schematic depicting a self-contained sandwich ELISA chip according to a preferred embodiment of the present invention.

### DETAILED DESCRIPTION OF THE PREFERRED EMBODIMENTS

Before the present invention is described in further detail, it should be understood that the invention is not limited to the particular embodiments described, and that the terms used in describing the particular embodiments are for the purpose of describing those particular embodiments only, and are not intended to be limiting, since the scope of the present invention will be limited only by the claims. Certain preferred embodiments applicable to particular applications will first be presented below in overview, and then in a more detailed description in conjunction with the drawings presented herein.

For some applications, it may be desirable to customize attributes of the electrochemical actuator for disposability. Disposable actuators would need to be made of inexpensive materials and be amenable to high-throughput manufacture. The inventors have made key steps in realizing disposability of electrochemical actuators, including snap-together, molded pump bodies; lower cost actuation electrodes (such as titanium), alternative pumping fluids and single-use, self-powered cells.

Electrochemical actuation can be used in concert with magnetic force to repeatedly open or close a fluidic path as presented in certain preferred embodiments. The fluid force of an electrochemical actuator can be used to push a metal rod into a fluidic path such that the path is blocked to flow of fluid. Then once it is desirable to initiate flow the electrochemically actuated fluid force can be reversed and a force (such as magnetic or vacuum) used to pull the metal rod back out of the fluidic path.

The fluid force of an electrochemical actuator can be used to repeatedly close a fluidic path in certain preferred embodiments by pressing upon an elastic membrane that bulges to block the path. By reversing the direction of the fluid force, the elastic membrane will contract away from the fluidic path to allow for flow. Electrochemically actuated fluid force pressing against a membrane can also be used to push fluid from a filled reservoir or to draw fluid into an empty reservoir. Additionally, electrochemical actuation can be used to drive a piston that is used to amplify the volume or pressure of fluid delivered.

Certain preferred embodiments encompass a wide array of programmable microfluidic chips such that all (or some) of the aspects of fluid movement are controlled by electrochemical actuation. In certain preferred embodiments, a bank of electrochemically actuated elastic valves is positioned along one or both sides of a fluidic channel, and sequential activation is used to move fluid peristaltically along a programmed path. One complicated embodiment for the coordinated use of a number of electrochemical actuators would be a single use, disposable chip for a sandwich ELISA. Key enabling features of a disposable ELISA chip would be on-board positive and negative controls for each test, disposal after one use of all components that come in contact with the sample, and snap fit into electrochemically actuated pump engines and electronic controls/readout.

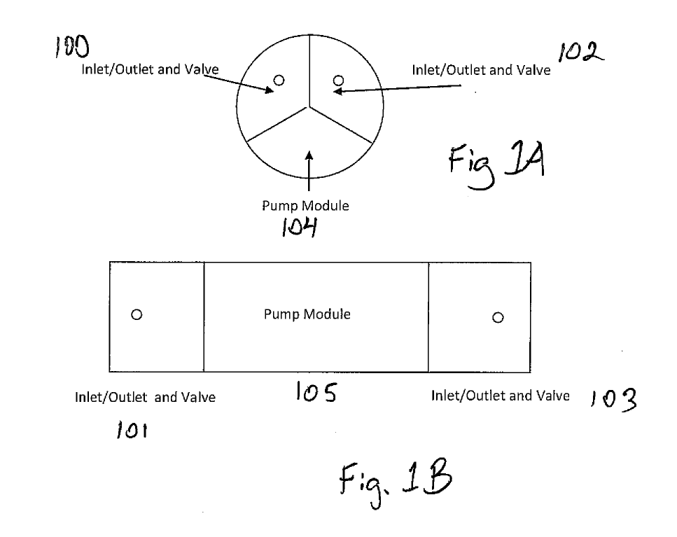

Turning now to a more specific discussion of the various applications in connection with preferred embodiments of the present invention, an electrochemically-actuated fluid delivery device incorporates electrochemical valves and electrochemical pumps within the same device. FIGS. 1A and 1B present a basic schematic of the electrochemical chamber arrangement for two different designs according to preferred embodiments. FIG. 1A is a circular design, with the two valve chambers 100 and 102 being adjacent to each other and also each being adjacent to pump module 104, while FIG. 1B is a linear design where pump module 105 separates valve chambers 101 and 103. The size and shape of the valves and pump can vary depending upon the specific performance parameters desired for a particular application. The inlet valve and the outlet valve can be physically identical, or can be of different shapes and sizes. The direction of fluid motion through the pump can be varied depending on operation of the pump. More specifically, either valve can be an inlet or an outlet. In fact, fluid can be both drawn in and pushed out of the same side of the pump. In the preferred embodiment to be used for drug infusion, however, one side of the pump (inlet valve) will be in fluidic contact with an external drug reservoir, and the other side (outlet valve) will be in fluidic contact with a patient (the application). The size, shape, number and volumes of the fluidic pathways and reservoirs can be specified for a particular application. The general structure and principles of operation related to the electrochemical valves and pump module may be understood from the discussion set forth in U.S. Pat. Nos. 7,718,047 and 7,909,101, which are incorporated by reference as if fully set forth herein.

The device of FIGS. 1A and 1B is an electrochemically-actuated fluid delivery device that operates on the coordinated actuation of electrochemical pumps and electrochemical valves. The result is a microfluidic device delivering nL/min-to-hundreds of μL/min flow rates with no mechanical parts. The direction of flow can be readily reversed by simply reversing the direction of the applied current/voltage.

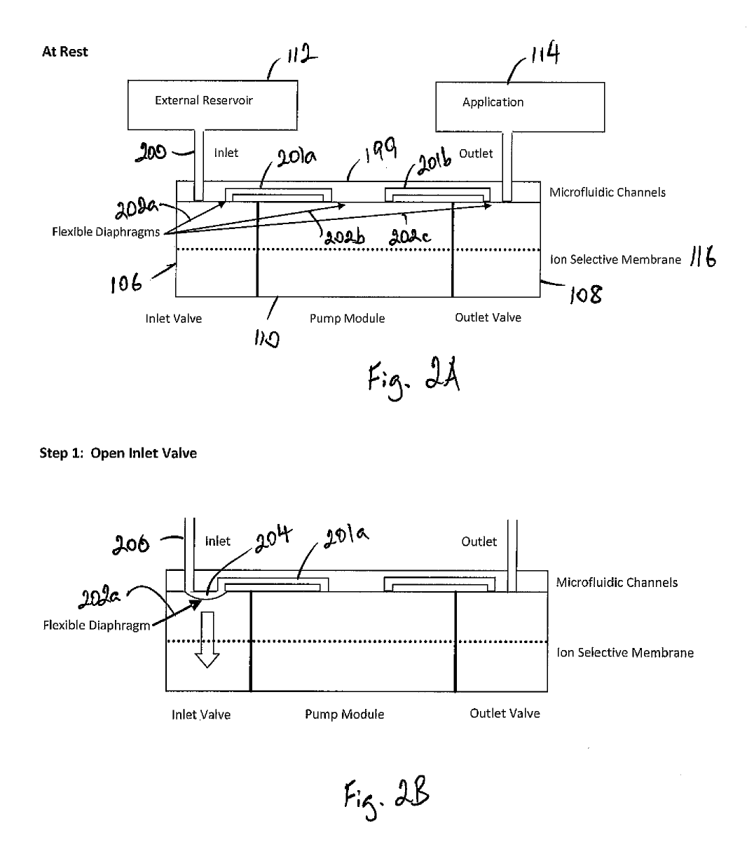

Referring now to FIG. 2A-2G, the structure and operation of a linear, “one-sided”embodiment of a single-dose fluid delivery pump may be described. One-sided in this case refers to the fact that there are fluid delivery connections on only one side of the ion selective membrane 116. Ion selective membrane 116 divides each of inlet valve 106, pump module 110, and outlet valve 108. A cap 199 is fitted over inlet valve 106, pump module 110, and outlet valve 108. In this case, cap 199 provides channels that are machined into cap 199 to provide fluid inlets and outlets. These fluid pathways allow fluid to flow from the external reservoir 112 into a flexible interior reservoir as will be described following, and from the internal reservoir to application 114. The two-valve system allows for carefully controlled delivery of fluid in intermittent or bolus doses and also acts as a dual-level safety feature to prevent accidental delivery of the entire contents of the external reservoir to the application in the case of a failure of the device. One example of a set of steps that will enable controlled drug delivery from external reservoir 112 to application 114 is detailed below, with reference to FIG. 2A showing the pump in the initial rest state, and FIGS. 2B-2G showing the sequential steps required to move fluid from external reservoir 112 to application 114.

When at rest as shown in FIG. 2A, the fluidic paths are blocked by the flexible diaphragms 202a, 202b, and 202c; diaphragm 202b is presented at pump module 110, diaphragm 202a is presented at inlet valve 106, and diaphragm 202c is presented at outlet valve 108. Because in the rest state the three diaphragms are not flexed, they effectively close the flow of fluid between external reservoir 112 and inlet channel 201a, between inlet channel 201a and outlet channel 201b, and between outlet channel 201b and application 114. In particular, flow of fluid between external reservoir 112 and inlet channel 201a is prevented by the fluidic pressure in inlet valve 106 pressing against diaphragm 202a. It may be seen that in this way, inlet valve 106 functions as a pinch valve.

Referring now to FIG. 2B, the first step in directional movement of fluid from external reservoir 112 is to electrochemically activate inlet valve 106, which causes pump fluid to move away from diaphragm 202a. This creates a vacuum, which pulls diaphragm 202a into a flexed position. As diaphragm 202a flexes, fluid is pulled from external reservoir 112 through inlet 200 into inlet pocket 204, which is created on the cap side of diaphragm 202a within inlet valve 106 as a result of the flexing of diaphragm 202a. In this way, inlet valve 106 now functions as a pinch valve that has been opened to allow flow into the device.

In the next step shown in FIG. 2C, pump module 110 is electrochemically activated, causing a vacuum that pulls diaphragm 202b into a flexed position. This action pulls fluid from inlet pocket 204, through inlet channel 201a, and then into internal reservoir 206 formed on the open side of diaphragm 202b. The result is that fluid is drawn from external reservoir 112 through inlet pocket 204 and inlet channel 201a into internal reservoir 206.

In the next step, shown in FIG. 2D, the voltage/current applied to inlet valve 106 is reversed, which causes diaphragm 202a to return to its rest (unflexed) position. It thus functions to close the valve previously created by inlet pocket 204. At this point, it is no longer possible for fluid in internal reservoir 206 to flow backward back to external reservoir 112.

In the next step shown in FIG. 2E, voltage/current is applied to outlet valve 108, thereby creating a vacuum that pulls diaphragm 202c into a flexed position. This creates outlet pocket 207, which opens a path between internal reservoir 206 and outlet 211, since internal reservoir 206 and outlet pocket 207 are connected by outlet channel 201b. Fluid from internal reservoir 206 will thus be allowed to flow into outlet channel 201b.

In the next step shown in FIG. 2F, the voltage/current at pump module 110 is reversed, which causes diaphragm 202b to return to its rest position. This causes internal reservoir 206 to shrink and in some cases eventually to disappear, which pushes the fluid from internal reservoir 206 through outlet channel 201b and outlet pocket 207, into outlet 211 and then on to application 114. The flow rate and volume/duration of fluid delivered can be controlled by controlling the applied voltage/current between the two separate electrochemical cells 209 and 210 in pump module 110. Although possible, it is not necessary to completely empty internal reservoir 206 prior to stopping flow. Additionally, it may be seen that overall pumping direction can be reversed to induce mixing of fluid delivered to application 114, as desired or required by specific fluids or fluid delivery applications.

In the final step shown in FIG. 2G, the appropriate voltage/current applied between the two separate chambers 212 and 213 of outlet valve 108 will cause diaphragm 202c to return to its rest position, thereby blocking the further flow of fluid to application 114. After the desired dose of fluid from internal reservoir 206 has been delivered in a continuous, intermittent, or bolus dose, the device should now be in the same position that it was at rest, as shown in FIG. 2A. Once again, each of the three chambers of the device is sealed from all others, and no fluid may flow between external reservoir 112 and application 114 in either direction. To move another dose of fluid from external reservoir 112 to application 114, the process herein described may be repeated. This process may be repeated as many times as desired for a particular application.

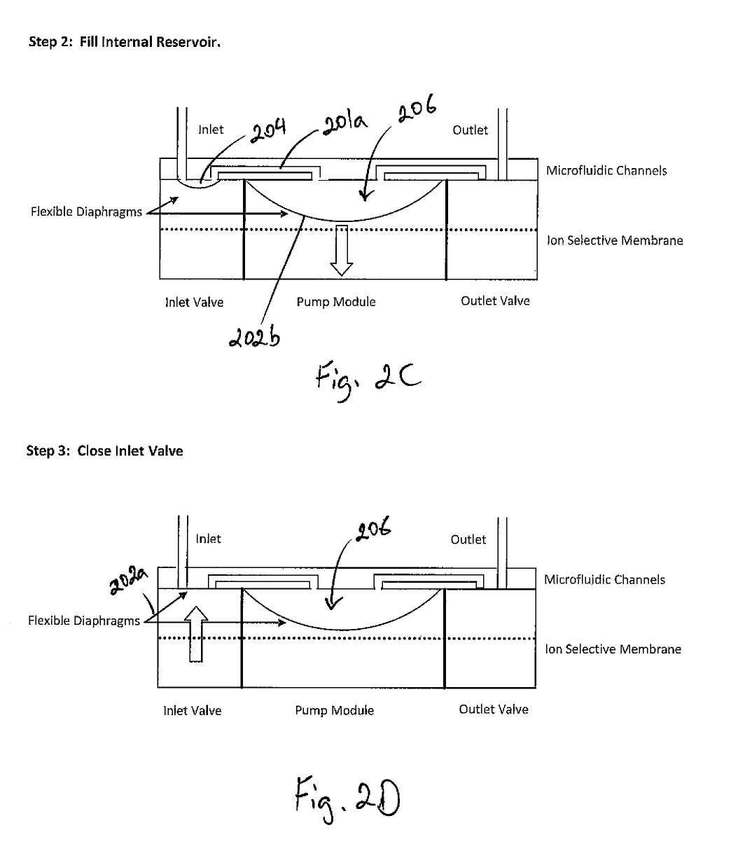

Referring now to FIG. 3A-3D, the structure and operation of a linear, “two-sided” embodiment of a continuous (or quasi-continuous) fluid delivery pump may be described. Two-sided in this case refers to the fact that there are fluid delivery connections on both sides of the ion selective membrane 316, first side 301 and second side 302. Ion selective membranes 316 divides each of inlet valve 326, pump module 328, and outlet valve 330. A first cap 332 and second cap 334 are fitted onto first side 301 and second side 302, respectively. Caps 332 and 334 feature machined channels that allow for the flow of fluid between inlets and outlets. These fluid pathways allow fluid to flow from external reservoir 312 into a flexible interior reservoir, and from the internal reservoir to application 314. One example of a set of steps that will enable controlled drug delivery from external reservoir 312 to application 314 is detailed below, with reference to FIG. 3A showing the pump in the initial rest state, and FIGS. 3B-3D showing the sequential steps required to move fluid from external reservoir 312 to application 314. In the case of this two-sided pump arrangement, the sub-steps described within each step may be performed simultaneously or in any order. At each step and/or at any time all valves and reservoirs can be either partially or fully opened or closed per requirements of the particular fluid delivery application, together with any mixing requirements.

When at rest as shown in FIG. 3A, all of the various fluidic paths are partially opened. Alternatively, the fluidic paths at first side 301 could be fully opened and the paths at second side 302 could be fully closed, or vice versa.

In the first step shown in FIG. 3B, inlet valve 326 is first activated such that force 1a is exerted at first inlet diaphragm 303, causing it to deflect within inlet valve 326. This causes the space adjacent diaphragm 303 to fill with fluid from external reservoir 312. This activation of inlet valve 326 also causes second inlet diaphragm 304 to deflect, which will close the space adjacent bottom inlet diaphragm 304, thereby stopping any flow of fluid from external reservoir 312 into that space. Next, pump module 328 is activated such that force 1b is exerted at first reservoir diaphragm 305, causing it to deflect within pump module 328. This causes the space adjacent diaphragm 305 to fill with fluid from external reservoir 312, since the path is now open between this space and external reservoir 312. This activation of pump module 328 also causes second reservoir diaphragm 306 to deflect, thereby stopping any flow of fluid into the space adjacent diaphragm 306.

In the next step shown in FIG. 3c, inlet valve 326 is first energized to exert force 2a. As a result, first inlet diaphragm 303 flexes to close the flow of fluid from external reservoir 312. In addition, this causes second inlet diaphragm 304 to move such that fluid may flow from external reservoir 312 into the space adjacent second inlet diaphragm 304. Next, outlet valve 330 is energized to exert force 2b, whereby first outlet diaphragm 307 is flexed to allow fluid to flow past it from adjacent first pump diaphragm 305 to application 314, and second outlet diaphragm 308 is flexed to block the flow of liquid from the area adjacent to second pump diaphragm 306 and then on to application 314. Then pump module 328 is energized to exert force 2c, whereby first pump diaphragm 305 is flexed to pump liquid from the area adjacent first pump diaphragm 305 toward first output valve 307, and block the flow of fluid from the area adjacent to first inlet diaphragm 303, and second pump diaphragm 306 is flexed to open the flow of fluid from the area adjacent to second inlet diaphragm 304 on to the area adjacent to second outlet diaphragm 308.

In the next step shown in FIG. 3D, the direction of each of the forces from FIG. 3C are reversed. Thus outlet valve 330 is first energized to exert force 3a. As a result, first outlet diaphragm 307 flexes to close the flow of fluid from the area adjacent to first pump diaphragm 305 to application 314. In addition, this causes second outlet diaphragm 308 to move such that fluid may flow from the area adjacent to second pump diaphragm 306 into the space adjacent second outlet diaphragm 308 and out to application 314. Next, inlet valve 326 is energized to exert force 3b, whereby first inlet diaphragm 303 is flexed to allow fluid to flow past it from external reservoir 312 to the area adjacent to first pump diaphragm 305, and second inlet diaphragm 304 is flexed to block the flow of liquid from external reservoir 312 to the area adjacent to second pump diaphragm 306. Then pump module 328 is energized to exert force 3c, whereby first pump diaphragm 305 is flexed to allow the entry of fluid from the area adjacent to first inlet diaphragm 303, and second pump diaphragm 306 is flexed to block the flow of fluid from the area adjacent to second inlet diaphragm 304 to the area adjacent to second outlet diaphragm 308.

It may be seen that be repeating the steps illustrated at FIGS. 3C and 3D, a continuous flow of fluid may be provided from external reservoir 312 to application 314, the flow alternating from taking a path along top 301 and along bottom 302 of the device. By manipulating the forces applied and the length of time that each step and sub-step is maintained, it may be seen that the fluid delivery rate can be customized per requirements of the application.

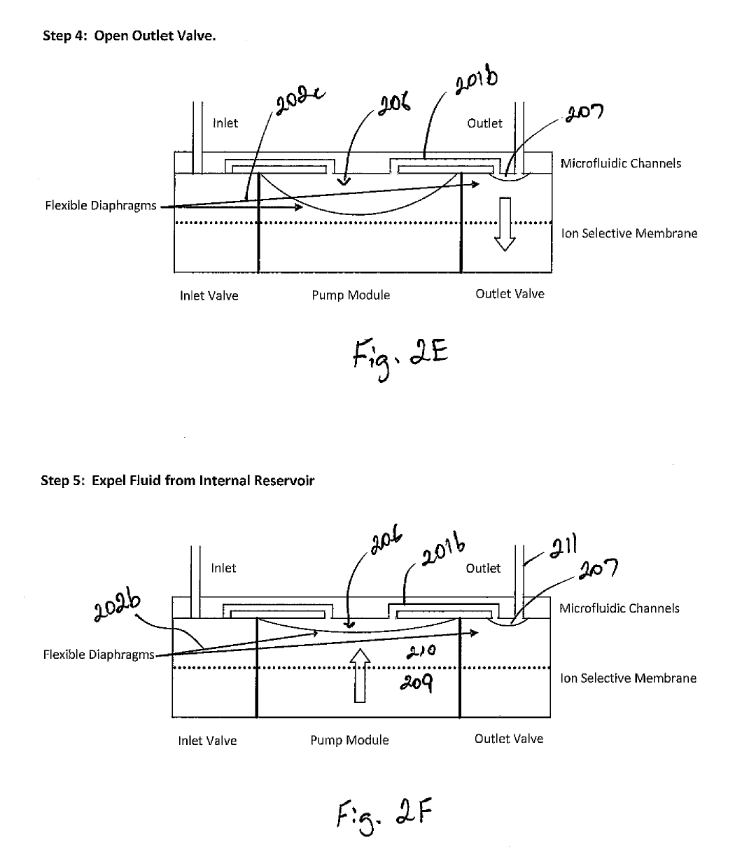

FIG. 4 shows experimental results from a quasi-continuous fluid delivery using electrochemical actuation to drive fluid flow. The experimental apparatus used two single-sided self-valving pumps arranged to simulate a dual-sided pump as set forth in FIGS. 3A-D. Dose 1 and 3 are delivered from the Side 301 Internal Reservoir and Dose 2 is delivered from the Side 302 Internal Reservoir (both corresponding to external reservoir 312 in the contemplated dual-sided embodiment). The noise produced during the gaps between doses is believed to be a result of the switching back and forth of the Side 301 and Side 302 Inlet and Outlet Valves, and can be greatly reduced by optimization of design and operation. In particular, the inventors believe that noise generated in FIG. 4 is a result of the retrofit modification of a single-sided pump to simulate a dual-sided pump, and that this noise would largely disappear in a true dual-sided embodiment.

The preferred embodiments of the fluid delivery devices as described herein provides a number of advantages to traditional pump/valve arrangements used for these applications. Fundamental engineering constraints limit the extent to which mechanical pumps can be miniaturized in order to meet the demands of certain applications, such as a wearable pump used for insulin delivery. In sharp contrast, the directional flow device described here is shape-independent, and requires very little power (typically on the order of mW) to deliver specific flow rates in the pL/min to μL/min flow rate range. More specifically, the device is operable for flow rates in the range of about 1 pL/min to about 100 μL/min, and to operate at a voltage of less than +/−2V. Flow precision is ±5% and dispense volumes as small as 100 nL have been delivered using this technology. Pumping pressures of up to about 300 psi may be achieved. The device according to preferred embodiments also offers truly pulse-free flow that is not normally possible with other pumps when continuous flow is desired. However, pulsed flow, as with bolus delivery of a single-dose of fluid, is also possible using electrochemical-actuation. The electrochemical fluidic action of the device allows it to open and close fluidic channels, acting as a valve as well as providing fluid flow, so that no mechanical valves or other external valving are required for operation.

The advantages of devices according to the preferred embodiment include the expansion of the variety of drugs that can be delivered via a wearable, patched or quasi-implantable device; being refillable during a simple outpatient or patient-administered procedure which is significantly less painful than injecting a new insert. Long-term, steady-state drug levels may lead to improved clinical outcomes; improved compliance and comfort for patients; and reduced number of visits to the physician, and hence reduced costs associated with the use of the device.

In a preferred embodiment, the device is formed from a non-reactive material such as polyetheretherketone (PEEK) plastic and assembled using standard fasteners. Other materials in alternative embodiments may be used, such as various plastic materials, including homo- or copolymers or their blends comprising, in addition to PEEK, polytetrafluoroethylene, polyethylene, polypropylene, polyesters, acrylic polymers, polyetherimide, polyamide, polyimide, and polyacetals. The device may be formed of parts that are, for example, machined or injection molded. The device may also in alternative embodiments be formed of one or more parts that comprise a coating of one material onto a different material, such as, for example, a Teflon coating over steel. In a preferred embodiment, a 2 μL target stroke volume of the device requires only 60 μL of pump fluid in each of the three chambers, limiting the overall size of the device to 180 μL, or 0.180 cm³. The low maximum flow rate of 2 μL/min requires the exposed area of ion selective membrane between any two chambers to be about 0.1 cm².

Various pumping protocols may be employed with the device according to a preferred embodiment. These will include giving small doses a couple of times a day, dispensing a larger bolus at desired intervals, continuous delivery, or programmable (staged and ramping rate) delivery. These can be adapted to fit each application. The voltage protocols may be optimized to customize the accuracy of dose delivery.

The control system (not shown in the figures) may consist of a battery along with all the hardware and firmware necessary for stand-alone control of the device, as will be readily understood by those skilled in the art. This controller may be connected to a computer, which will upload the specific pumping instructions into the controller's firmware. Then the controller may be removed from the computer and attached to the device, which will then begin the dispensing protocol. Alternatively, the controller can be operated wirelessly from a stationary controller or an enabled wireless communication device, such as a smartphone. The control system is preferably designed to be as small and light as feasible while still providing the voltage control and current necessary to drive the device. The controller also is preferably designed to minimize power requirements to increase battery life. An inductive recharging system may be used as an alternative. In the envisioned final commercial device, this controller may ultimately be hermetically sealed within the body of the device for reliable operation.

In the particular application of insulin delivery, it may be seen that the preferred embodiments described herein offer numerous advantages over existing devices for this purpose. Table 1 summarizes the differences between the device and certain commercially available insulin delivery devices, and also sets forth the advantages that the device offers over these existing commercial devices.

TABLE 1

| | | | | | Advantage of |
|---|---|---|---|---|---|
| | Present | Insulet | Animas ® One | Debiotech | Present |
| Features | invention | OmniPod ® | Touch ® Ping ™ | JewelPUMP ™ | Invention |
| Pump | Electrochemical: | SMA ratcheting | Syringe - | MEMS - | Simple battery |
| Mechanism | non mechanical | driver | stepper motor | piezoelectric | operation |
| | | (mechanical) | | actuated | |
| Pump | Adjustable | Alignment with 2 | Syringe | Single | Tunable Form |
| Shape/size | | mL syringe | alignment with | disposable | Factor |
| | | | reservoir | chip | |
| Basal Flow | Dictated by | 0.05 IU | 0.025 IU | 0.02 IU per | Pulseless flow |
| Limits | ability to control | Resolution | Resolution | Actuation | <0.025 IU/h |
| | power | | | | |
| Wireless | Yes | Yes | No | Yes | SmartPhone |
| Control | | | | | compatibility |
| Cost | Low | Low | Large up-front | Low | Very |
| | | | cost | | competitive |
| Pump | Adjustable, | 3 day, | Refill every few | 6 d, | Limited only |
| Lifetime | Disposable | Disposable | days | Disposable | by Insulin |
| | | | | | capacity |

There are several methods to enable disposability of electrochemical actuators, including a molded actuator body, use of less expensive (as compared to platinum) electrodes and self-powered battery-free operation. Example preferred embodiments of each of these attributes is provided herein.

### Molded Actuator Body

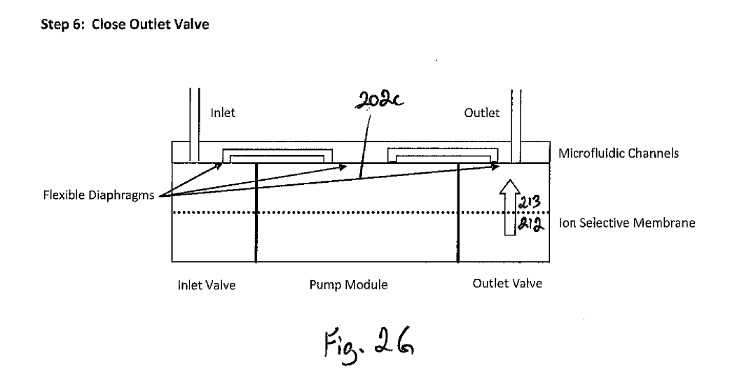

FIG. 5 is a drawing of the body of a molded electrochemical actuator according to a preferred embodiment of the present invention. A semi-permeable membrane 510 is sandwiched between two pump body pieces 506 and a leak-free fit ensured by gaskets 508. A diaphragm 504 is fitted out the outside of each of pump body pieces 506, and a pump cap 502 is fitted into place over each diaphragm 504. These parts can be easily assembled, for example, by overmolding, inlay molding, ultrasonic welding, snap-fit, and screw fit, which are methods that are amenable to high-throughput, low-cost molding and assembly.

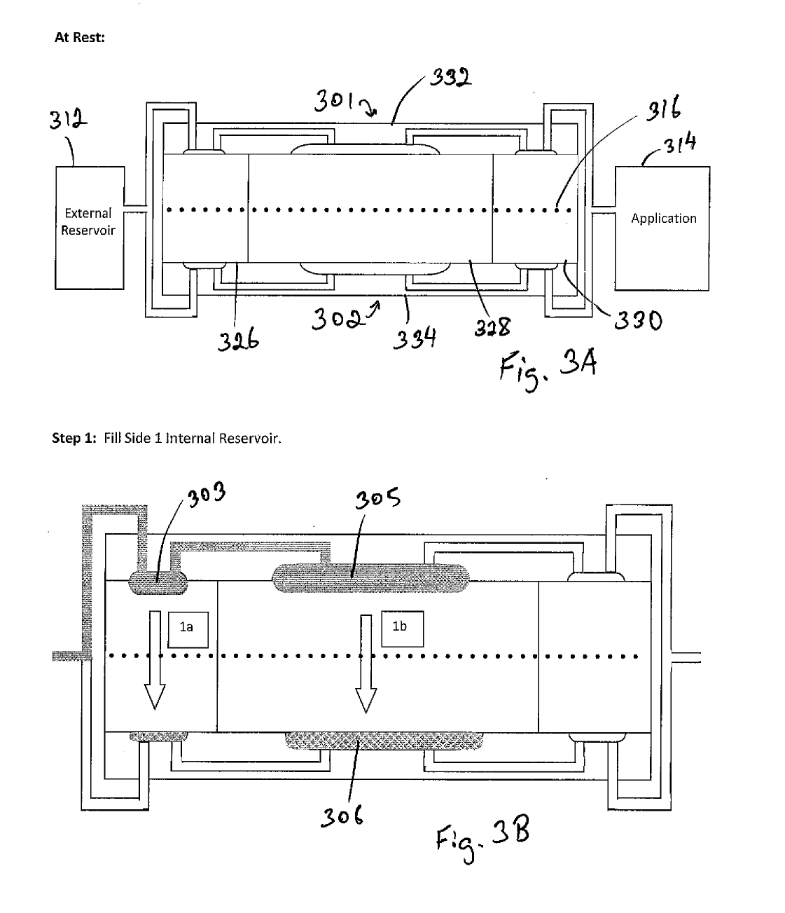

Traditionally, electrochemical actuation has been conducted using a high end electrode material, such as platinum. However, the inventors have found that lower cost materials, such as titanium and palladium, can also be used as electrodes in electrochemical actuators according to a preferred embodiment. FIG. 6 shows the electrochemical current produced in 20 mM ferrocyanide and ferricyanide, with 0.1M potassium chloride using titanium electrodes in the actuator body. The electrochemical current produces a flux of ions that drives fluid flow within the actuator.

Electrochemical actuation can be driven by a galvanic chemical reaction in which a potential difference is created between the halves of the actuator body upon creation of an electrical connection between the cathode in one half of the cell with the anode in the other half of the cell. An ion flux is generated across the membrane that separates the two halves of the actuator body; this ion flux will continue until either the anode or its corresponding electrolyte is exhausted. Thus, the reaction is in only one direction and is irreversible. The ion flux drives fluid flow within the actuator.

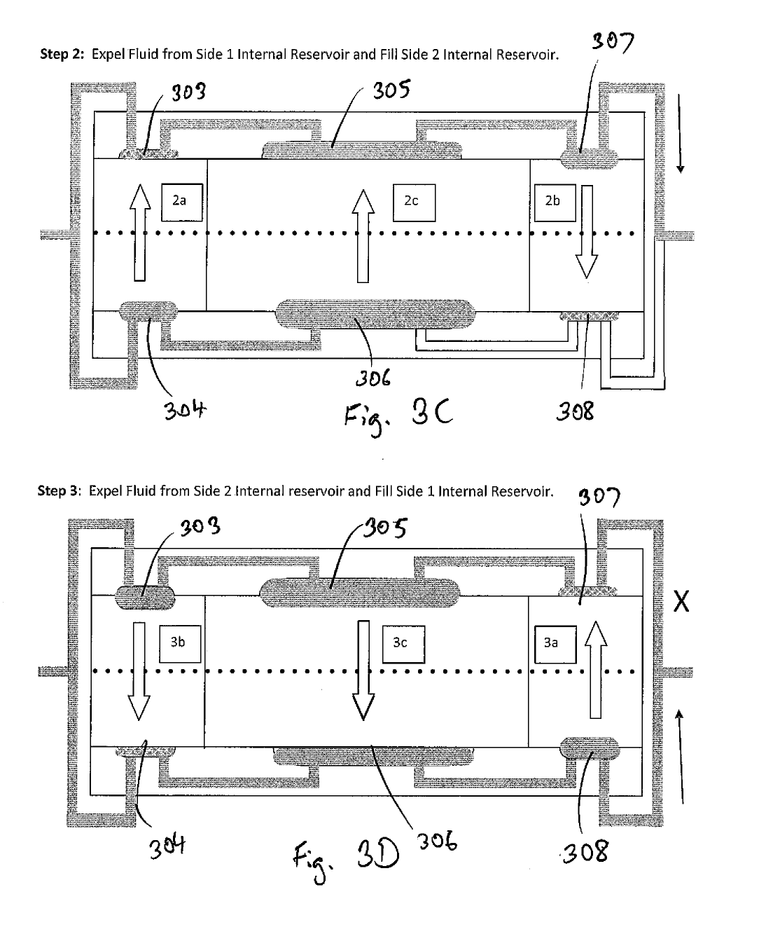

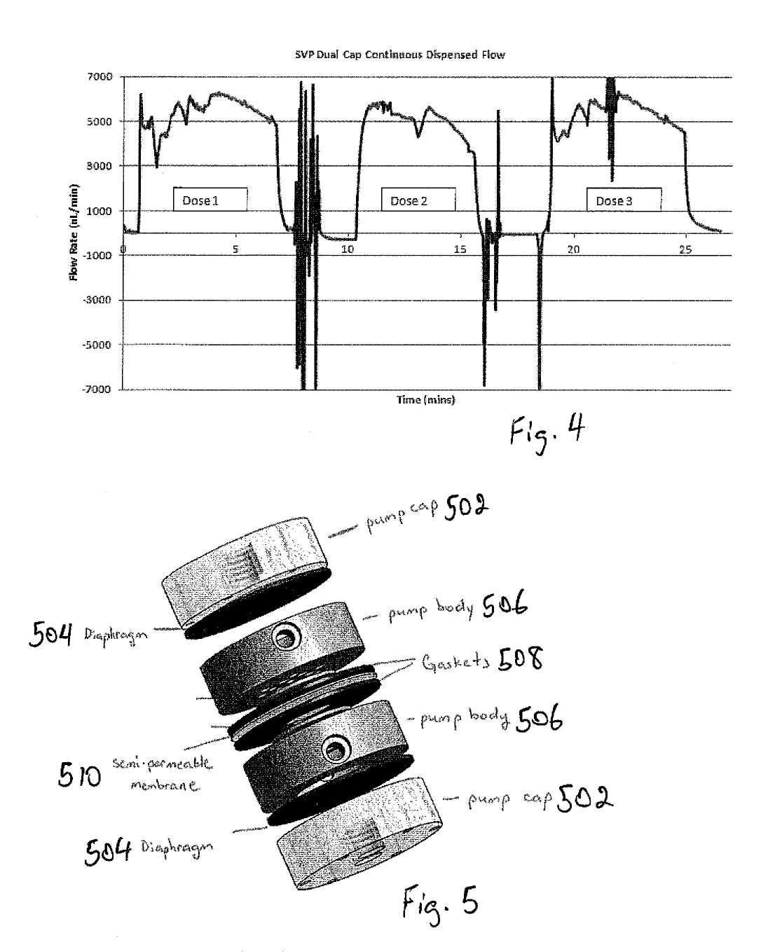

FIGS. 7A and 7B show one embodiment of a galvanic electrochemical actuator. In this embodiment, platinum is used as the cathode 704 and iron is used as the anode 702. Prior to actuation the chemistry in both halves of the cell 700 are at equilibrium and fluid flow is not produced. Once the anode 702 (Fe, Cu, Al or other metal) and cathode 704 (Pt, Ti or other metal) are connected electrically, the unequal potential difference between the two halves of the actuator induces a flux of Li⁺ (or other) cations across the membrane 706. This flux of ions will continue until the iron anode 702 is fully oxidized or the ions are depleted. The ion flux will expand the flexible diaphragms 708 and generate fluid flow from the right side of the actuator as shown in FIG. 7B. FIG. 8 (Top) shows the electric current produced by the self-powered electrochemical actuator and the resultant flow rate of fluid. The top line and left hand y-axis are the fluid flow rate and the bottom line and right hand y-axis are the corresponding electric current. FIG. 8B shows a linear increase in the volume of fluid delivered over time showing very steady fluid delivery using the galvanic electrochemical actuator. Both flow rate, as shown in FIG. 8A, and duration of the generation of flow can be customized to a particular application by specifying the size of the anode that is oxidized (in this case iron) during the reaction or by specifying the concentration of species that is reduced (in this case iodine to iodide) in that cell of the actuator body.

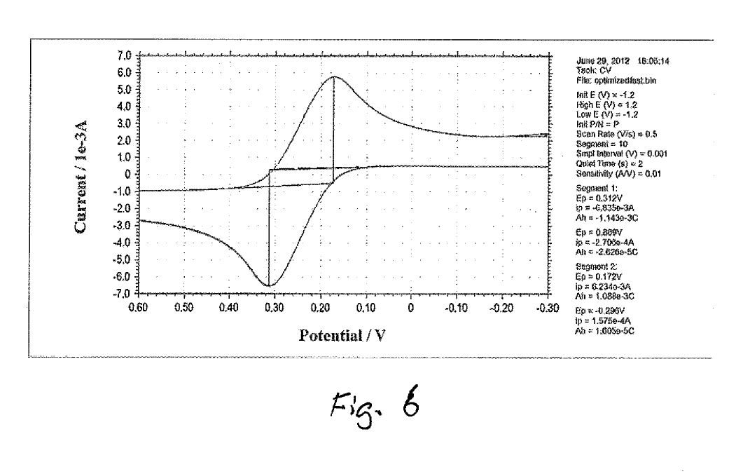

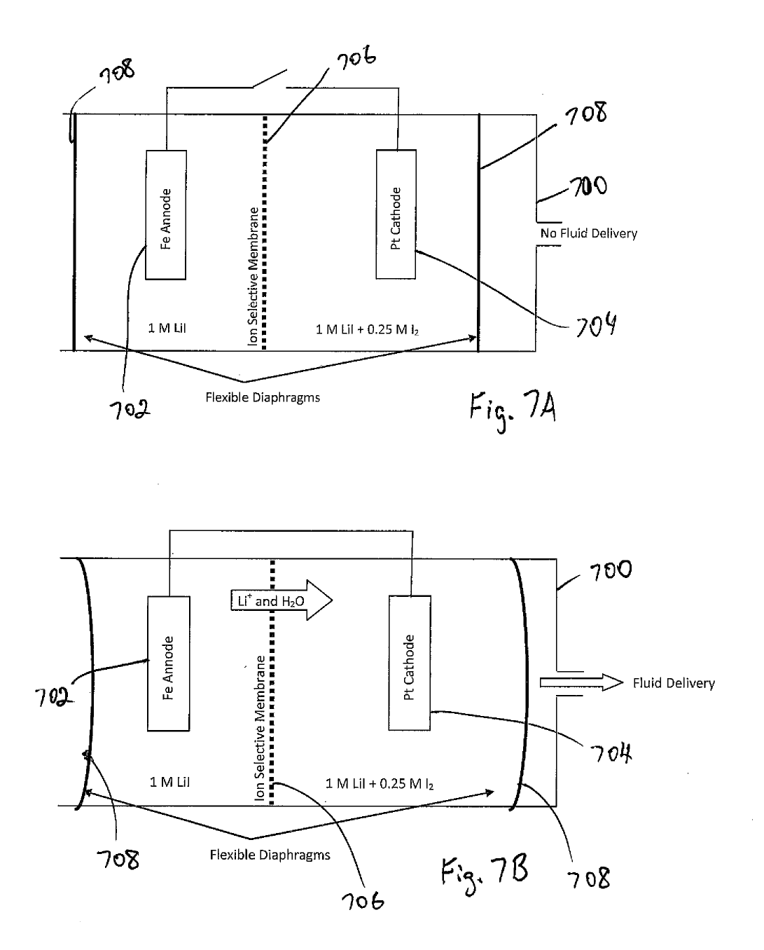

The fluid pumped by an electrochemical actuator can be used to engage or disengage a magnetic valve, as shown in FIG. 9. As shown in inset A, fluid flow is used to expand an elastic diaphragm 902 away from magnetic 900, which then pushes a cylindrical metal valve 904 into a fluidic path to block flow. Using the actuator, fluid flow can then be reversed such that pressure is taken off the elastic diaphragm 902 and the metal valve 904. The magnet 900 above the valve 904 will then pull the metal valve 904 up and out of the fluidic path to enable flow. A setup as in FIG. 9 inset B was used to demonstrate this embodiment of magnetic valve actuation. As shown in FIG. 10, when the magnetic valve 904 is closed using the actuator, flow is through the Upchurch flow sensor 910 only, and no flow is sensed at the Sensirion flow sensor 908. And, when the magnetic valve 904 is open (at time=13 minutes), fluid flow proceeds through both sensors.

Another mechanism by which an electrochemical actuator can be used for microfluidic flow control involves direct contact of an elastic diaphragm on an actuator with an elastic diaphragm within a microfluidic network. Because fluid flow within the actuator can be easily and repeatedly reversed, the actuator can be used multiple times to perform the same function. In this embodiment, flow is run in one direction to actuate the device; then the (voltage/current) is reversed, and the resultant flow is reversed to reset the device. The elastic diaphragm on the actuator can be in contact with another elastic diaphragm on a reservoir for dispense/aspirate functions or on a channel itself for pinch valve/pump applications.

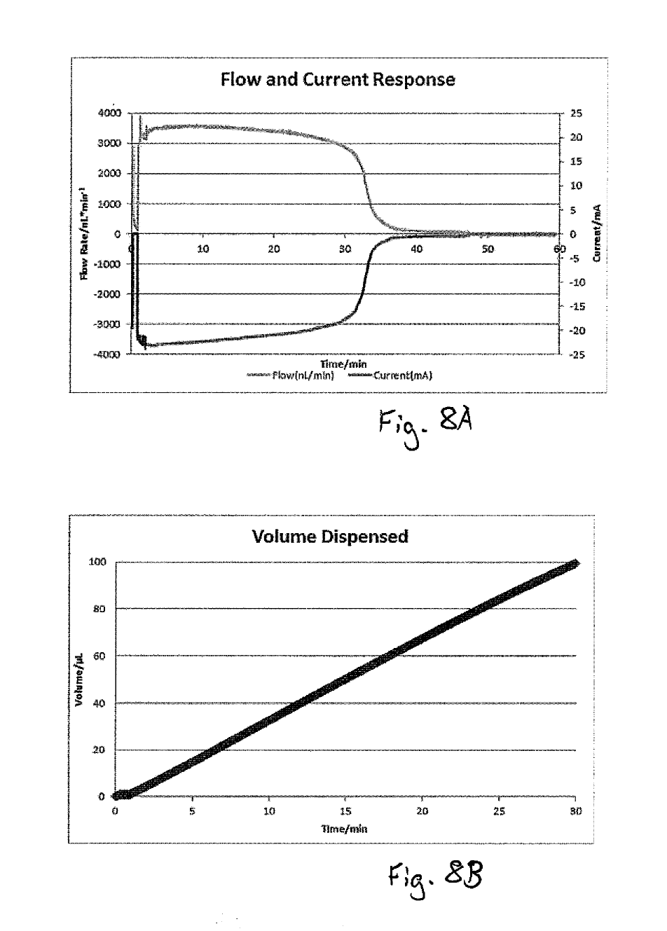

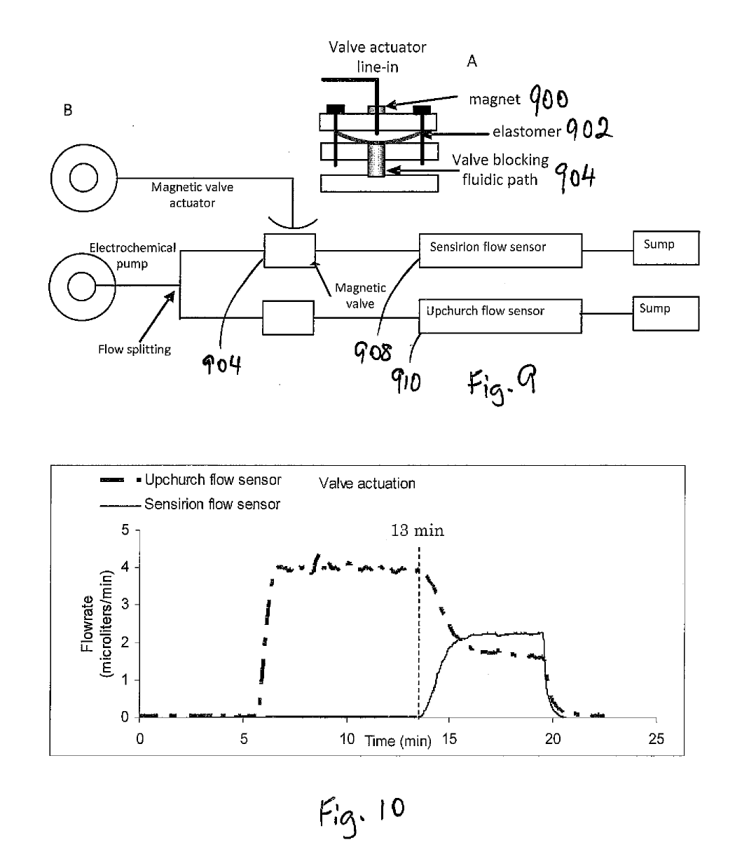

FIG. 11 shows how an electrochemical actuator can be used to dispense fluid from or aspirate fluid into a reservoir. In order for the device to operate, the elastomer diaphragm 1102 must be in contact with the elastomer reservoir cover 1104 on the microfluidic chip 1108. If the reservoir is initially full, electrochemical actuation within the actuator could push the diaphragm 1102 outward against the reservoir cover 1104 which would then push fluid from the reservoir into the microfluidic channel 1106. FIG. 12 shows experimental electrochemical actuation flow control from a reservoir using this method of actuation. Flow can be controlled at specified flow rates using diaphragm to diaphragm actuation. If the reservoir is initially empty, electrochemical actuation could pull the diaphragm away from the reservoir and allow fluid to enter the reservoir. If a vacuum is created between the two flexible membranes or if the elastomer reservoir cover is designed to return to the unflexed state, the fluid will be actively drawn into the reservoir.

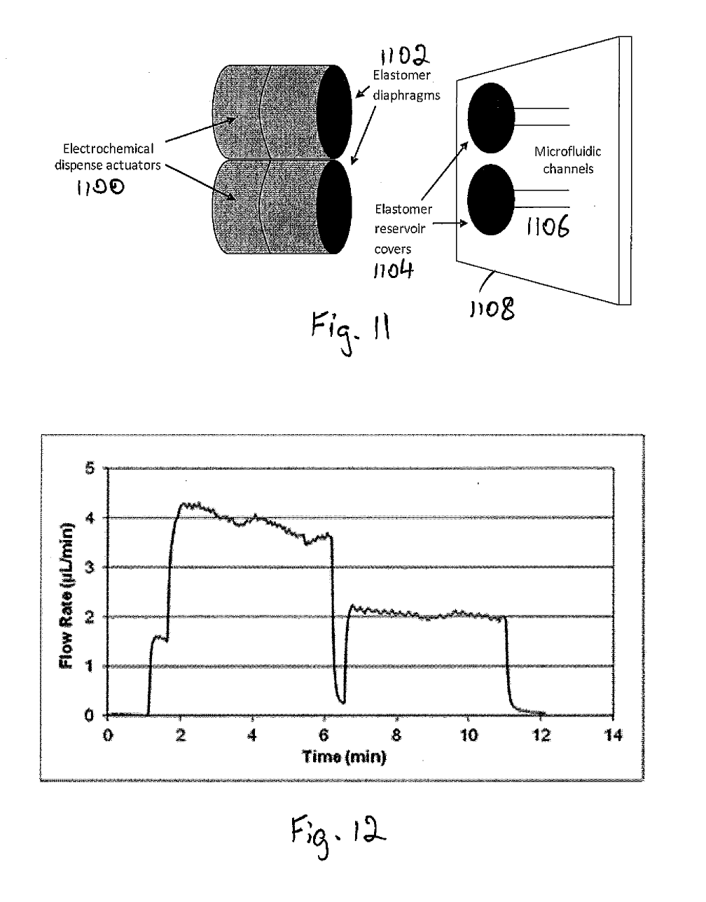

This same concept can be used to engineer precise flow control (both pulsed and pulseless) within a microfluidic network. A bank of diaphragm actuators along a fluidic path could be used to open one segment of a path to allow fluid in. The next segment of the path can be opened (partially or fully) while the first segment is being closed (partially or fully). In this manner, fluid could be moved along preprogrammed paths peristaltically. The preprogrammed path could be multidirectional and branching to meet any number of specifications for flow control. FIGS. 13A through 13E demonstrate just a few of a multitude of embodiments wherein the path is actuated electrochemically as a pinch pump/valve. Each X in FIG. 13A could be the location of an external actuator wherein the flexible diaphragm is in contact with (or actually constitutes) a flexible section of the channel. If the pinch valve/pump is open, fluid flow is enabled. If the pinch valve/pump is closed, fluid flow is blocked. FIG. 13B shows how flow from two paths can be merged continuously into one path. FIG. 13C shows how fluid from two paths can be merged intermittently into one path. FIG. 13D shows how direction of flow can be intermittently reversed to induce mixing of the two fluids. FIG. 13Ee shows how mixing can be induced by transport of fluids through an “S” bend. This is not intended to be a comprehensive list of actions that can be performed by this embodiment, rather to give an idea of the range of operations that could be conducted on such a device.

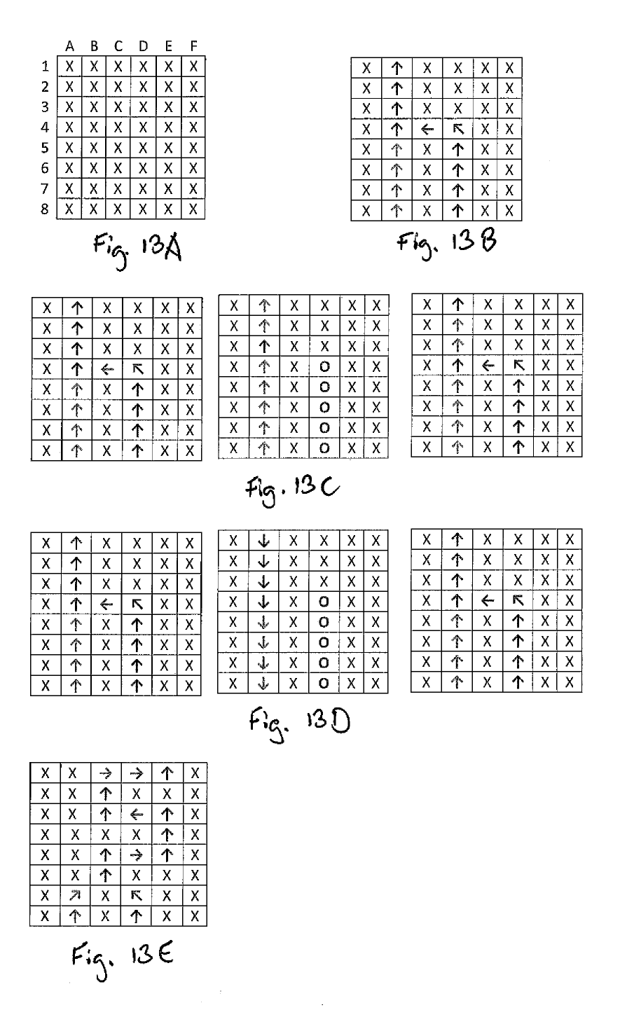

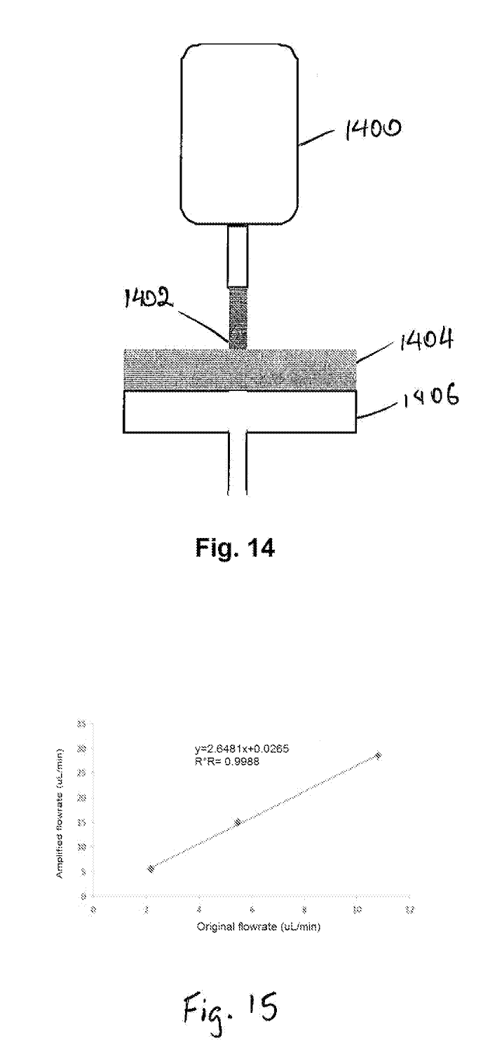

Electrochemical actuation can be used to drive a piston that is used to either increase the volumetric flow rate of fluid or increase the pressure at which fluid is delivered. In FIG. 14, the diaphragm of an electrochemical actuator 1400 is in direct or fluidic contact with the small end of a piston 1402. The other end of the same piston has a greater surface area 1404 and is in contact with an elastic membrane of a correspondingly larger reservoir 1406. Electrochemical actuation can then be used to drive the piston forward and expel a proportionally greater amount of fluid per unit time from the reservoir 1406, resulting in either a higher volumetric flow rate or larger dispense volume in a given time interval (see FIG. 15).

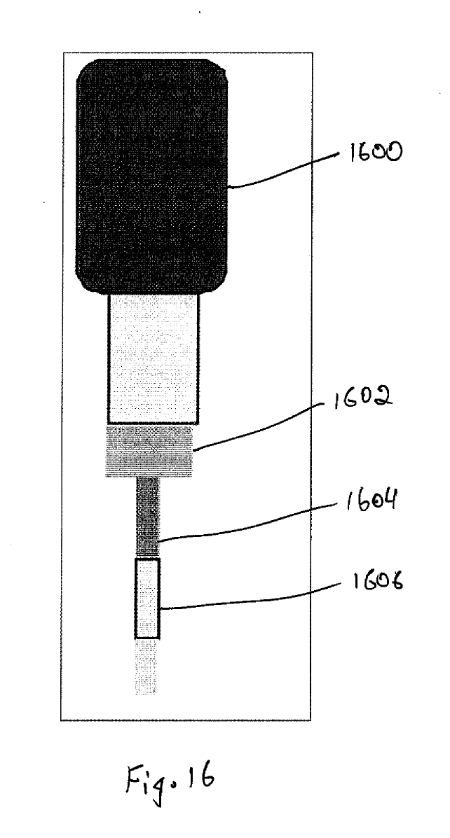

In FIG. 16, the diaphragm of an electrochemical actuator 1600 is in direct or fluidic contact with the large end of a piston 1602. The smaller end 1604 of the same piston is in contact with an elastic membrane of a correspondingly smaller reservoir 1606. Electrochemical actuation can then be used to drive forward the piston and expel a proportionally smaller amount of fluid from the reservoir 1606. In this setup, the pressure that the fluid is pumped at will increase proportionally to the decrease in volume of fluid, and the device is a pressure amplification unit.

In order to return the actuator, piston and reservoir membrane to their original positions, electrochemical actuation would be operated in the opposite direction by supplying current/voltage in the opposite direction. The actuator is then ready to perform another amplification stroke. As shown previously in FIG. 3, a dual-sided electrochemical actuator in concert with electrochemical valves could be used to drive one or two pistons. The coordinated operation of this device could be used to continuously or quasi-continuously deliver fluids at amplified volumes or pressures.

In addition to varying the surface area of the pistons, the relative viscosity of the fluid on either side of the piston could be adjusted to customize flow and operation. For example, if a highly viscous fluid were used as the working fluid in the pump, leakage and evaporation from around the piston will be reduced.

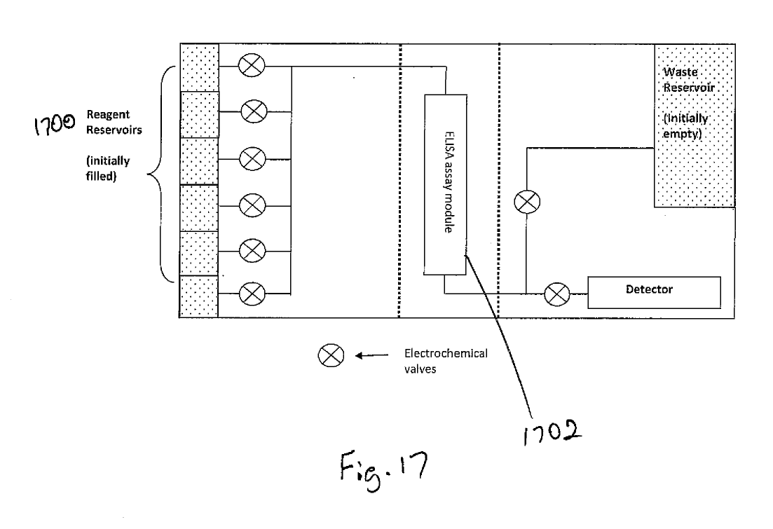

FIG. 17 is an embodiment of a disposable sandwich ELISA microfluidic chip that uses many of the electrochemical actuators that have been previously presented. The entire chip shown in this figure could be disposed of after a single use. This would eliminate the possibility of between-sample cross contamination. The small size of this chip and use of inexpensive materials would make disposability both practical and affordable. In this example, the bank of six reagent reservoirs 1700 would allow for on-chip storage of wash solution, secondary antibody solution, report molecule solution, positive control, negative control and sample solution. One side of each reservoir could be a flexible membrane that would come into contact with a bank of corresponding electrochemical actuators with diaphragm to diaphragm contact. As needed, the electrochemical pinch valves 1704 could be actuated to open a channel and allow fluid to be expelled from each reservoir individually in accordance with a desired protocol. The sandwich ELISA would be built within the assay module 1702 and all spent reagents could be pushed or aspirated into the waste reservoir via controlled electrochemical actuation. The use of pinch valves 1704 at the exit of the assay module would allow only the solution with the reporter molecule to pass over the detector to eliminate the possibility of prior contamination with waste reagents. This example is not meant to limit the applicability of this technology to ELISA only, but rather is intended to demonstrate the precise on-chip flow control that is enabled by electrochemical actuation in one particular preferred embodiment.

Certain ranges have been provided in the description of these particular embodiments with respect to certain parameters. When a range of values is provided, it should be understood that each intervening value between the upper and lower limit of that range and any other stated or intervening value in that stated range is encompassed within the invention, subject to any specifically excluded limit in the stated range. Where the stated range of values includes one or both of the limits, ranges excluding either or both of those limits are also included in the scope of the invention.

Unless otherwise stated, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein. It will be apparent to those skilled in the art that many more modifications are possible without departing from the inventive concepts herein. All terms used herein should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. As used herein, “consisting of” excludes any element, step, or ingredients not specified in the claim element. As used herein, “consisting essentially of” does not exclude materials or steps that do not materially affect the underlying novel characteristics of the claim. When a Markush group or other grouping is used herein, all individual members of the group and all combinations and subcombinations possible of the group are intended to be individually included in the disclosure. All references cited herein are hereby incorporated by reference to the extent that there is no inconsistency with the disclosure of this specification.

The present invention has been described with reference to certain preferred and alternative embodiments that are intended to be exemplary only and not limiting to the full scope of the present invention as set forth in the appended claims.

1. A device for the directional delivery of a fluid, comprising:
a. a pump module comprising an electrochemical actuator configured to selectively apply a pressure within the pump module;
b. an inlet valve comprising an electrochemical actuator configured to selectively apply a pressure within the inlet valve;
c. an outlet valve comprising an electrochemical actuator configured to selectively apply a pressure within the outlet valve;
d. an external reservoir in fluid communication with the inlet valve;
e. an application in fluid communication with the outlet valve;
f. a first channel fluidically connecting the inlet valve and the pump module;
g. a second channel fluidically connecting the outlet valve and the pump module;
h. an inlet diaphragm positioned within the inlet valve, wherein the inlet diaphragm is flexible to assume one of a flexed state and an unflexed rest state, and wherein the inlet diaphragm is positioned to block flow of the fluid between the external reservoir and the first channel when the diaphragm is in the unflexed (or flexed) state, and to allow flow of the fluid between the external reservoir and the first channel when the inlet diaphragm is in the flexed (or unflexed) state;
i. an outlet diaphragm positioned within the outlet valve, wherein the outlet diaphragm is flexible to assume one of a flexed state and an unflexed rest state, and wherein the outlet diaphragm is positioned to block flow of the fluid between the second channel and the application when the diaphragm is in the unflexed rest state, and to allow flow of the fluid between the second channel and the application when the diaphragm is in the flexed state; and
j. an internal reservoir diaphragm positioned within the pump module, wherein the internal reservoir diaphragm is flexible to assume one of a flexed state and an unflexed rest state, and wherein the internal reservoir diaphragm is positioned to receive flow of the liquid from the first channel into a reservoir formed by the internal reservoir diaphragm when the internal reservoir diaphragm is in the flexed state, and to discharge the fluid from the reservoir into the second channel when the internal reservoir diaphragm moves from the flexed state to the unflexed state.

2. The device of claim 1, further comprising an external reservoir in fluid communication with the external reservoir inlet.

3. The device of claim 2, wherein the application outlet is fluidically connected to an application.

4. The device of claim 3, wherein the fluid comprises a medically therapeutic fluid.

5. The device of claim 4, wherein the fluid comprises insulin.

6. The device of claim 1, further comprising an electronic control system in electrical communication with the inlet valve, pump module, and outlet valve and configured to selectively activate and deactivate each of the inlet valve, pump module, and outlet valve.

7. The device of claim 6, wherein the electronic control system is further operable to selectively reverse the direction of operation of each of the inlet valve, pump module, and outlet valve.

8. The device of claim 6, further comprising a sensor in communication with the electronic control system and operable to send an electrical signal in response to an external stimuli, wherein the electronic control system is configured to activate and deactivate each of the inlet valve, pump module, and outlet valve in response to the electrical signal.

9. The device of claim 8, wherein the sensor is a glucose sensor and the fluid comprises insulin.

10. The device of claim 1, wherein each of the inlet valve, pump module, and outlet valve comprise an electrolyte solution, and wherein the inlet valve diaphragm, internal reservoir diaphragm, and outlet valve diaphragm are impermeable with respect to the electrolyte solution.

11. The device of claim 1, wherein a voltage to selectively apply a pressure within at least one of the inlet valve, pump module, and outlet valve is less than 2 V.

12. The device of claim 1, wherein the device is configured to deliver the fluid at a rate in the range of 1 pL/min to 1 mL/min.

13. The device of claim 1, further comprising:
a. A second external reservoir inlet in fluid communication with the inlet valve;
b. A second application outlet in fluid communication with the outlet valve;
c. a second inlet channel fluidically connecting the inlet valve and the pump module;
d. a second outlet channel fluidically connecting the outlet valve and the pump module;
e. a second inlet diaphragm positioned within the inlet valve, wherein the second inlet diaphragm is flexible to assume one of a flexed state and an unflexed rest state, and wherein the second inlet diaphragm is positioned to block flow of the fluid between the second external reservoir inlet and the second inlet channel when the second inlet diaphragm is in the unflexed rest state, and to allow flow of the fluid between the second external reservoir inlet and the second inlet channel when the second inlet diaphragm is in the flexed state;
f. a second outlet diaphragm positioned within the outlet valve, wherein the second outlet diaphragm is flexible to assume one of a flexed state and an unflexed rest state, and wherein the second outlet diaphragm is positioned to block flow of the fluid between the second outlet channel and the second application outlet when the second outlet diaphragm is in the unflexed (or flexed) state, and to allow flow of the fluid between the second outlet channel and the second application outlet when the second outlet diaphragm is in the flexed (or unflexed) state; and
g. a second internal reservoir diaphragm positioned within the pump module, wherein the second internal reservoir diaphragm is flexible to assume one of a flexed state and an unflexed rest state, and wherein the second internal reservoir diaphragm is positioned to receive flow of the liquid from the second inlet channel into a second reservoir formed by the second internal reservoir diaphragm when the second internal reservoir diaphragm moves from an unflexed state to a flexed state, and to discharge the fluid from the second internal reservoir into the second outlet channel when the second internal reservoir diaphragm moves from the flexed state to the unflexed state.

14. The device of claim 13, further comprising an electronic control system in electrical communication with the inlet valve, pump module, and outlet valve, wherein the electronic control system is configured to selectively activate and deactivate each of the inlet valve, pump module, and outlet valve and to selectively reverse the direction of operation of the inlet valve, pump module, and outlet valve.

15. The device of claim 14, further comprising a sensor in communication with the electronic control system and operable to send an electrical signal in response to an external stimulus, wherein the electronic control system is configured to selectively activate and deactivate and to selectively reverse the direction of operation of at least one of the inlet valve, pump module, and outlet valve in response to the electrical signal.

16. A method of directionally pumping a fluid using a pump, wherein the pump comprises a pump module, an inlet valve, an outlet valve, an external reservoir inlet in fluid communication with the inlet valve, an application outlet in fluid communication with the outlet valve, an inlet channel fluidically connecting the inlet valve and the pump module, an outlet channel fluidically connecting the outlet valve and the pump module, an inlet diaphragm positioned within the inlet valve, an outlet diaphragm positioned within the outlet valve, and an internal reservoir diaphragm positioned within the pump module, the method comprising the steps of:
a. activating the inlet valve to create a force in a first direction, whereby the inlet diaphragm flexes in the first direction to form an inlet passage between the external reservoir inlet and the inlet channel;
b. activating the pump module to create a force in the first direction, whereby the internal reservoir diaphragm flexes in the first direction to form an internal reservoir, and thereby drawing the fluid from the external reservoir inlet into the internal reservoir;
c. activating the inlet valve to create a force in a second direction, whereby the inlet diaphragm returns to a rest state to close the inlet passage between the external reservoir inlet and the inlet channel;
d. activating the outlet valve to create a force in the first direction, whereby the outlet diaphragm flexes in the first direction to form an outlet passage between the outlet channel and the application outlet; and
e. activating the pump module to create a force in the second direction, whereby the internal reservoir diaphragm flexes in the second direction, thereby forcing fluid from the internal reservoir through the outlet channel and into the application outlet.

17. The method of claim 16, further comprising the step of activating the outlet valve to create a force in the second direction, whereby the outlet diaphragm returns to a rest state to close the outlet passage between the outlet channel and the application outlet.

18. The method of claim 17, wherein the step of activating the outlet valve and a pump module to create a force in the second direction further comprises the step of pumping the fluid from the application outlet to a mammalian body.

19. The method of claim 16, further comprising the step of mixing the fluid with a second fluid by selectively activating at least one of the inlet valve, pump module, and outlet valve.

20. The method of claim 16, wherein a voltage required to activate at least one of the inlet valve, pump reservoir, and outlet valve is less than 2 V.

21. The method of claim 16, wherein the step of activating the pump module to create a force in the second direction, whereby the internal reservoir diaphragm flexes in the second direction, thereby forcing fluid from the internal reservoir through the outlet channel and into the application outlet, comprises the step of delivering the fluid into the application outlet at a rate in the range of 1 pL/min to 1 mL/min.

22. A method of directionally pumping a fluid using a pump, wherein the pump comprises a pump module, an inlet valve, an outlet valve, a first external reservoir inlet and a second external reservoir inlet in fluid communication with the inlet valve, a first application outlet and a second application outlet in fluid communication with the outlet valve, a first inlet channel and a second inlet channel fluidically connecting the inlet valve and the pump module, a first outlet channel and a second outlet channel fluidically connecting the outlet valve and the pump module, a first inlet diaphragm and a second inlet diaphragm positioned within the inlet valve, a first outlet diaphragm and a second outlet diaphragm positioned within the outlet valve, and a first internal reservoir diaphragm and a second internal reservoir diaphragm positioned within the pump module, the method comprising the steps of:
a. activating the inlet valve to create a force in a first direction, whereby the first inlet diaphragm flexes in the first direction to form a first inlet passage between the first external reservoir inlet and the first inlet channel, and the second inlet diaphragm flexes in the first direction to close a second inlet passage between the second external reservoir inlet and the second inlet channel;
b. activating the pump module to create a force in the first direction, whereby the first internal reservoir diaphragm flexes in the first direction to form a first internal reservoir, and thereby drawing the fluid from the first external reservoir inlet into the first internal reservoir;
c. activating the inlet valve to create a force in a second direction, whereby the first inlet diaphragm returns to a rest state to close the inlet passage between the first external reservoir inlet and the first inlet channel, and whereby the second inlet diaphragm flexes in the second direction to form a second inlet passage between the second external reservoir inlet and the second inlet channel;
d. activating the outlet valve to create a force in the first direction, whereby the first outlet diaphragm flexes in the first direction to form a first outlet passage between the first outlet channel and the first application outlet;
e. activating the pump module to create a force in a second direction, whereby the first internal reservoir diaphragm flexes in the second direction, thereby forcing fluid from the first internal reservoir through the first outlet channel and into the first application outlet, and whereby the second internal reservoir diaphragm flexes in the second direction, thereby drawing fluid from the second inlet channel into the second internal reservoir;
f. activating the inlet valve to create a force in the first direction, whereby the first inlet diaphragm flexes in the first direction to open the first inlet passage between the first external reservoir inlet and the first inlet channel, and the second inlet diaphragm flexes in the first direction to close the second inlet passage between the second external reservoir inlet and the second inlet channel;
g. activating the outlet valve to create a force in the second direction, whereby the first inlet valve diaphragm flexes in the second direction to close the first outlet passage between the first outlet channel and the first application outlet, and the second outlet diaphragm flexes in the second direction to open the second outlet passage between the second outlet channel and the second application outlet; and
h. activating the pump module to create a force in the first direction, whereby the first internal reservoir diaphragm flexes in the first direction to open the first internal reservoir, and thereby drawing the fluid from the first external reservoir inlet into the first internal reservoir, and whereby the second internal reservoir diaphragm flexes in the first direction, thereby forcing fluid from the second internal reservoir through the second outlet channel and into the second application outlet.

23. The method of claim 22, wherein the steps of activating the outlet valve and pump module to create a force in the second direction and activating the outlet valve to create a force in the first direction further comprise the step of pumping the fluid from at least one of the first application outlet and second application outlet to a mammalian body.

24. The method of claim 22, further comprising the step of mixing the fluid with a second fluid by selectively activating at least one of the inlet valve, pump module, and outlet valve.

25. The method of claim 22, wherein a voltage required to activate at least one of the inlet valve, pump reservoir, and outlet valve is less than 2 V.

26. The method of claim 22, wherein the steps of activating the outlet valve to create a force in the second direction and activating the outlet valve to create a force in the first direction each comprise the step of delivering the fluid into at least one of the first application outlet and the second application outlet at a rate in the range of 1 pL/min to 100 μL/min.

27. An electrochemical actuator, comprising:
a. a semi-permeable membrane comprising a first and second side;
b. a first pump body positioned adjacent to the first membrane side;
c. a second pump body positioned adjacent to the second membrane side;
d. a first diaphragm positioned adjacent to the first pump body opposite from the semi-permeable membrane;
e. a second diaphragm positioned adjacent to the second pump body opposite from the semi-permeable membrane;
f. a first pump cap positioned adjacent to the first diaphragm; and
g. a second pump cap positioned adjacent to the second diaphragm.

28. The electrochemical actuator of claim 27, wherein the first and second pump body and first and second pump cap each comprise one of machined parts and injection molded parts.

29. The electrochemical actuator of claim 28, further comprising a first gasket positioned between the semi-permeable membrane and the first pump body, and a second gasket positioned between the semi-permeable membrane and the second pump body.

30. The electrochemical actuator of claim 29, wherein at least one of the first and second pump body and first and second pump cap are formed of a material selected from the set consisting of polyetheretherketone, polyethylene, polypropylene, polyester, acrylic polymer, polyetherimide, polyamide, polyimide, and polyacetal.

31. The electrochemical actuator of claim 29, wherein at least one of the first and second pump body and first and second cap comprise a base material covered with a coating material.

32. The electrochemical actuator of claim 31, wherein the coating material comprises Teflon.

33. The electrochemical actuator of claim 32, wherein the base material comprises steel.

34. The electrochemical actuator of claim 28, wherein at least two of the first and second pump body and first and second pump cap are connected by one of ultrasonic welding, snap-fit placement, screw-fit placement, overmolding, inlay molding, injection molding, or fasteners.

35. The electrochemical actuator of claim 27, wherein at least one of the first and second pump body comprise an electrode, and wherein the electrode is formed from a material selected from the group consisting of titanium, palladium, a titanium-coated material, a palladium-coated material, and carbon.

36. A valve actuator in communication with a fluidic path, comprising:
a. an electrochemical pump comprising an elastomer;
b. a mechanical valve in contact with the elastomer, whereby the mechanical valve is operable to open and close the fluidic path; and
c. a force generator configured to generate a force capable of moving the mechanical valve against the elastomer in a direction opposite of a direction in which the elastomer expands as a result of operation of the electrochemical pump.

37. The valve actuator of claim 36, wherein the mechanical valve is one of a magnet and a ferromagnetic material, and wherein the force generator is one of a magnet and a ferromagnetic material.

38. A fluid control mechanism, comprising:
a. a flexible fluid container comprising a volume; and
b. an electrochemical actuator comprising an elastomer diaphragm, wherein the electrochemical actuator is configured to flex the elastomer diaphragm outward, and further wherein the elastomer diaphragm is in contact with the fluid container whereby outward flexing of the elastomer diaphragm reduces the volume of the fluid container.

39. The fluid control mechanism of claim 38, wherein the electrochemical actuator is used in conjunction with one of a magnetic force, a magnetohydrodynamic force, an ultrasonic force, an electrohydrodynamic force, an electroosmotic force, an electrokinetic force, a piezoelectric force, an osmotic force, a peristaltic force, and a motorized force.

40. The fluid control mechanism of claim 38, wherein the fluid container is a fluid reservoir, and wherein outward flexing of the elastomer diaphragm presses against a wall of the fluid reservoir thereby forcing fluid from the fluid reservoir through an exit channel.

41. The fluid control mechanism of claim 40, wherein the elastomer diaphragm is connected to the fluid container and the electrochemical actuator is further configured to flex the elastomer diaphragm inward, whereby inward flexing of the elastomer diaphragm increases the volume of the fluid container.

42. The fluid control mechanism of claim 41, wherein the fluid container is a fluid reservoir, and wherein inward flexing of the elastomer diaphragm increases the volume of the fluid reservoir, thereby aspirating a fluid into the fluid reservoir from an entry channel.

43. The fluid control mechanism of claim 38, wherein the fluid container is a flow channel, and whereby outward flexing of the elastomer diaphragm closes the flow channel thereby blocking the flow of fluid through the flow channel.

44. The fluid control mechanism of claim 43, comprising a plurality of electrochemical actuators configured to operate in unison.

45. The fluid control mechanism of claim 44, wherein the fluid control mechanism is operable to mix a plurality of fluids within the flow channel.

46. The fluid control mechanism of claim 43 comprising a plurality of electrochemical actuators each in communication with one of a plurality of reagent reservoirs, and further comprising an assay module in fluid communication with each of the electrochemical actuators such that any reagent from the plurality of reagent reservoirs may be delivered to the assay module.

47. An actuation device, comprising:
a. an electrochemical actuator comprising an elastomer diaphragm, wherein the electrochemical actuator is configured to flex the elastomer diaphragm outward and wherein the electrochemical actuator comprises a diaphragm cross-sectional area;
b. a piston comprising a first and second section wherein the piston first section is connected to the elastomer diaphragm; and
c. a flow channel sized to receive the second section of the piston.

48. The actuation device of claim 47, wherein the piston first section comprises a first cross-sectional area equal to the diaphragm cross-sectional area and the piston second section comprises a second cross-sectional area different from the first cross-sectional area.

49. The actuation device of claim 47, wherein the device further comprises an actuation fluid comprising an actuation fluid viscosity and within the electrochemical actuator and bounded by an elastomer diaphragm, a piston or a barrier comprised of a solid, a liquid, or a gas, wherein the flow channel comprises a pumped fluid comprising a pumped fluid viscosity, and wherein the actuation fluid viscosity is not equal to the pumped fluid viscosity.

50. A galvanic electrochemical actuator, comprising:
a. a first electrochemical cell half comprising an electrolyte and a cathode;
b. a second electrochemical cell half comprising the electrolyte and an anode;
c. an ion-permeable membrane separating the first and second electrochemical cell halves; and
d. an electrical connection between the cathode and anode, whereby an ion flux is generated through the ion permeable membrane.

51. The galvanic electrochemical actuator of claim 50, wherein the cathode comprises platinum and the anode comprises iron.
